(19) United States
(12) Patent Application Publication
(10) Pub. No.: US 2016/0207853 A1
(43) Pub. Date: Jul. 21, 2016

(54) EXTRACTIVE DISTILLATION PROCESS

(71) Applicant: BLUE CUBE IP LLC, Midland, MI (US)

(72) Inventors: Bruce S. Holden, Midland, MI (US); Jacob M. Crosthwaite, Midland, MI (US); Robert M. Campbell, Midland, MI (US)

(21) Appl. No.: 14/914,542

(22) PCT Filed: Aug. 27, 2014
(86) PCT No.: PCT/US2014/052832

§ 371 Date: Feb. 25, 2016

Related U.S. Application Data
(60) Provisional application No. 61/871,384, filed on Aug. 29, 2013.

(51) Int. Cl. C07C 7/08; B01D 3/40

(52) U.S. Cl. CPC C07C 7/08; B01D 3/40

(57) ABSTRACT

A process for extracting divinylarene from a composition mixture stream comprising at least an ethylvinylarene and at least a divinylarene, the process comprising the steps of: (a) contacting a composition mixture stream comprising at least an ethylvinylarene and at least a divinylarene with a liquid extractive agent such that at least a portion of the divinylarene present in the composition mixture is extracted into the liquid extractive agent; and (b) recovering at least a portion of the divinylarene extracted from the ethylvinylarene stream to form a divinylarene product stream; wherein the divinylarene product stream contains at least 50 weight percent divinylarene; and an apparatus for extracting divinylarene from the composition mixture stream.

### FIELD

The present invention is related to an extractive distillation process and apparatus therefor; and more specifically, to an extractive distillation process and apparatus therefor for removing or separating a divinylarene from an ethylvinylarene.

### BACKGROUND

Divinylarenes such as divinylbenzene (DVB) are important raw materials used in the production of other important downstream products. Downstream products made from for example a divinylarenes such as DVB are beneficially used in many applications including for example coatings, composites, laminates and adhesives.

As discussed further hereinafter, typically during the production of DVB, a crude DVB material has to undergo a separation process, currently a conventional distillation process is practiced in the art as the separation process, to separate a desired DVB product from undesired byproducts or impurities such as ethylvinylbenzene (EVB) and diethylbenzene (DEB) that may be present in the crude DVB material. The separation process is preferred to produce a DVB raw material which is substantially free of the above undesired byproducts and impurities, i.e., a pure DVB raw material.

By increasing the purity of DVB, less waste is produced resulting in a more efficient process. In addition, by separating and isolating an undesired byproduct such as EVB, the EVB can be recycled back to a DVB reactor, thus improving the yield of the DVB process. Also, a pure DVB product is desirable for use in downstream products.

One known process to produce DVB is catalytic dehydrogenation of diethylbenzene (DEB). Using the current catalyst and reaction conditions of about 550° C.-600° C. and 350 mmHg absolute, a portion (about ⅔) of the DEB is only partially (about 75%) dehydrogenated, resulting in the coproduction of roughly equivalent quantities of DVB and EVB in residual DEB and reaction byproducts. Generally, a DVB/EVB ratio of about 1.0 is found in the dehydrogenation reactor effluent (referred to as “crude DVB”) using the above known process. A purer DVB, i.e., a product having a DVB/EVB ratio of about 1.22-4.0 is generally desired in the final product to obtain a DVB product with better properties. Therefore, prior art crude DVB produced by known processes requires a DVB purification step to obtain a purer DVB product.

Currently, DVB purification is done by fractional distillation under vacuum to keep temperatures low (e.g., below 110° C.) and to minimize polymerization losses of DVB and EVB, i.e., about 5% or lower. However, the relative volatility between EVB (the light) and DVB (the heavy) is low at typical distillation temperatures, in the range of 1.3-1.5. This requires a distillation tower with many (e.g., >30) theoretical stages and a significant amount of reflux (e.g., >3:1 reflux ratio) which results in a relatively large and costly distillation tower for a given capacity that has a high energy consumption per pound of product (e.g., >1.5 lb steam/lb finished product). In addition, a large distillation tower tends to result in high reboiler temperatures and long retention times (more than approximately 1 hour) which contribute to excessive polymer formation rates.

Conventional fractional distillation is the principal known process that is used to perform the DVB-EVB separation. However, liquid-liquid extraction is considered as a lower-cost, lower-energy method of performing the separation.

Laboratory fractional extraction experiments have been carried out in which DVB is separated out of crude DVB using triethylene glycol (TEG) and n-heptane as dual solvents in a 51 mm diameter packed pulsed extractor. The above experiments show that in a 4.6 m tall column, a 90 weight percent (wt %) DVB purity is achieved in the extract on an EVB-DVB basis with 80 percent (%) recovery of DVB out of the raffinate. However, the above laboratory results are obtained at a total throughput of only 0.57 m³/h/m² so phase contact times are quite long. Scale-up of the laboratory process to a commercial liquid-liquid extractor at such a low throughput is not economically feasible. Recent mini-plant column liquid-liquid extraction experiments on stripped crude DVB with TEG and n-octane show more reasonable throughputs of 24 m³/h/m². However, the results of the mini-plant experiments show low separation efficiencies which require a number of commercial-scale liquid-liquid extractors in series to achieve the required number of theoretical stages of separation.

Heretofore, liquid-liquid extraction of DVB from EVB with DMSO (dimethyl sulfoxide) and n-heptane has also been considered by the prior art wherein the separation factor (essentially the equilibrium driving force for the extraction) is so low that a very large number of theoretical stages is required for the separation, also rendering that option infeasible.

Extractive distillation has been used in several other applications, notably in light hydrocarbons plants for aromatics/aliphatics separations where extractive distillation has largely replaced the use of liquid-liquid extraction for new hydrocarbons plants. For example, extractive distillation is used in light hydrocarbon plants for aromatic/aliphatic separation. Extractive distillation has also been used for decades to separate butanes, butenes, and butadiene and is a known process for separating olefins from paraffins in general. A good review of extractive distillation is in the article “Distillation, Azeotropic and Extractive” in Kirk-Othmer Encyclopedia of Chemical Technology (vol. 8) as well as in Perry's Chemical Engineers' Handbook 8^{th} Edition, Don W. Green (2008), pg 13-87 to 93. It is also discussed in Handbook of Separation Techniques for Chemical Engineers, Philip A. Schweitzer (1979), pages 1-135 to 143.

The prior art is replete with references teaching the use of extractive distillation. However, no prior art is found that discloses extractive distillation of divinylarenes, particularly the use of extractive distillation to separate DVB from EVB. For example, U.S. Pat. No. 5,523,502 discloses flexible light olefins production and describes extractive distillation of olefins from paraffins resourced from steam cracking and catalytic cracking processes. However, the process of the above patent does not address extractive distillation of aromatic compounds or divinylarene compounds. The above patent also does not disclose extractive agents, such as sulfolane, which are operable for extractive distillation of aromatic compounds or divinylarene compounds.

U.S. Pat. No. 5,750,798 discloses a method for making ether from a paraffin feedstock and describes extractive distillation of olefins from paraffins, particularly separating butenes from butanes using a variety of possible extractive agents. However, the above patent does not disclose separating aromatic or divinylarene compounds using extractive distillation.

U.S. Pat. No. 7,699,962 discloses a process utilizing extractive distillation to separate components of a reactor effluent. The above patent mentions DVB as one of a number of possible monomers used in a polymerization reactor with hydrofluorocarbons. However, the process of the above patent is restricted to extractive distillation of C4-C7 isoolefins from hydrofluorocarbons. The above patent does not disclose or teach the use of extractive distillation to separate divinylarenes from ethylvinylarenes.

DVB and EVB compounds are aromatic and have olefinic character. And, DVB and EVB are much more similar in chemical structure and properties than, for example, butanes and butenes. For example, the only difference between DVB from EVB is that EVB is lacking one of two C—C double bonds present in DVB. Thus, it is not apparent to the skilled artisan that an extractive agent that is useful for separating butenes from butanes is effective in separating DVB from EVB because DVB and EVB are very similar in structure; and this similarity makes it difficult to separate the two compounds.

### SUMMARY

The present invention is directed to the use of an extractive distillation process to separate divinylarene from ethylvinylarene. The present invention extractive distillation process enhances the relative volatility between EVB and DVB (as well as DEB and DVB) with an extractive agent with a relative volatility to DVB that is less than 1.0 and which has a preferential affinity for DVB over EVB. Thus, DVB and EVB compounds which are similar in chemical structure and properties can be effectively separated from one another using the process of the present invention.

In one embodiment, the present invention includes a process for separating divinylarene from ethylvinylarene including the steps of:

(a) contacting an ethylvinylarene stream containing divinylarene with a liquid extractive agent such that at least a portion of the divinylarene present in the ethylvinylarene stream is extracted into the liquid extractive agent; and

(b) recovering the divinylarene extracted from the ethylvinylarene.

In another embodiment, the present invention includes an apparatus for extracting divinylarene from an ethylvinylarene including:

(a) means for contacting an ethylvinylarene stream containing divinylarene with a liquid extractive agent such as for example a distillation vessel; and

(b) means for recovering the divinylarene extracted from the ethylvinylarene such as a recovery vessel connected to the contacting means of (a).

One of the objectives of the present invention is to enhance the relative volatility between EVB and DVB sufficient to significantly reduce the size of the distillation tower used in the process for separating DVB from EVB and DEB and reduce the size of the required reboiler and condenser duties; resulting in a significant reduction in the energy consumption of the present process. The reduction in energy, in turn, would result in a reduction in capital; and a reduction in operating costs for the present invention process. For example, no additional new DVB separation train would be required; and/or no additional capacity for an existing distillation train in a DVB plant would be required using the present invention process.

For example, extractive distillation of DVB from EVB and DEB has the advantage of lower energy consumption and lower capital costs than conventional distillation. Preliminary engineering of a conventional DVB-EVB distillation tower for a 50 kTa DVB plant resulted in a 35 ft (10.5 m) diameter tower with 110 ft (33 m) of packing requiring 71.3 MM Btu/h (20,906 kW) of reboiler duty. For a sulfolane extractive distillation using sulfolane, for example, a process simulation of the DVB-EVB separation predicted that in the same 50 kTa DVB plant the DVB-EVB extractive distillation tower to achieve the same product specifications would only require a 16 ft (4.9 m) diameter tower with 90 ft (27.4 m) of packing requiring only 13.5 MM Btu/h (3950 kW) of reboiler duty, an 80% energy reduction.

The use of a liquid-liquid extraction process to separate DVB from EVB and DEB also results in lower energy consumption than the conventional distillation process, but an extractive distillation process is much less complex than a liquid-liquid extraction process because no aliphatic wash solvent (e.g., n-octane) is required which eliminates the need to use several distillation towers to recover and purify the wash solvent for reuse in the extraction.

The above simulations are supported by laboratory vapor-liquid equilibrium measurements on DVB/EVB/DEB/sulfolane mixtures compared to DVB/EVB/DEB mixtures without sulfolane. The relative volatility of EVB to DVB at about 80° C. in the absence of sulfolane is only 1.45, and the relative volatility of EVB to DVB, at about 80° C. and at a sulfolane to DVB/EVB/DEB ratio of 1.5, is boosted to 2.0 relative volatility which is a significant increase of the relative volatility. At a higher sulfolane to DVB/EVB/DEB ratio of 2.5, the relative volatility of EVB to DVB at about 80° C. is even higher, i.e., a 2.1 relative volatility.

Relative volatility is an indicator of the ease of separation of components in a mixture by distillation. For example, the greater the value of relative volatility of a composition above 1.0, the easier it is to separate components in a mixture by distillation. Therefore, a relative volatility of 2 of a composition containing sulfolane results in a smaller tower being required (less diameter and height) than is required when distilling DVB, EVB, and DEB without the presence of sulfolane.

### BRIEF DESCRIPTION OF THE DRAWINGS

For the purpose of illustrating the present invention, the drawings show a form of the present invention which is presently preferred. However, it should be understood that the present invention is not limited to the embodiments shown in the drawings.

FIG. 1 is a schematic diagram of a DVB extractive distillation concept showing a distillation apparatus with incoming and outgoing streams.

FIG. 2 is a schematic process flow diagram showing an example of a process utilizing an extractive distillation apparatus and process of the present invention.

### DETAILED DESCRIPTION

One broad embodiment of the present invention is directed to a vapor-liquid extractive distillation process for separating DVB from EVB including the steps of:

(a) contacting an EVB stream containing DVB with a liquid extractive agent such that at least a portion of the divinylarene present in the ethylvinylarene stream is extracted into the liquid extractive agent;

(b) recovering the DVB extracted from the EVB; and

(c) recovering the extractive agent from the EVB of step (b) which has been depleted of DVB.

The distillation process of the present invention may be carried out in an extractive distillation tower at a predetermined temperature and a predetermined pressure; and an extractive distillation tower having a predetermined number of theoretical equilibrium stages sufficient to remove DVB from EVB (see Perry's Chemical Engineers' Handbook 8^{th} Edition, Don W. Green (2008), pg. 13-5 for a discussion on theoretical equilibrium stages). The conditions of the process may vary depending on the extracting agent used in the process. For example, the temperature of the distillation process may be generally from about 45° C. to about 160° C. in one embodiment; from about 55° C. to about 140° C. in another embodiment; and from about 65° C. to about 125° C. in still another embodiment.

Generally, the pressure of the process may be from about 0.006 atmospheres (atm) to about 0.07 atm in one embodiment, between about 0.01 atm to about 0.04 atm in another embodiment, and between about 0.013 atm to about 0.026 atm in still another embodiment.

The number of theoretical stages required in the extractive distillation tower to achieve the desired separation of DVB from EVB will depend on the degree of separation desired as well as the degree of enhancement of the EVB/DVB relative volatility provided by the particular extractive agent used in the process. In general, the number of theoretical equilibrium stages for the process may be chosen between about 5 to about 100 in one embodiment, between about 15 to about 60 in another embodiment, and between about 25 to about 45 in still another embodiment. Below a stage count of about 5, the amount of separation between DVB and EVB is insufficient such that the process becomes uneconomical; and above a stage count of about 100, the size of the distillation equipment is so large, relative to the degree of separation between DVB and EVB, that the process is not practical or economical.

The steps of the process of the present invention include contacting a composition with (i) at least one liquid extractive agent in a distillation tower.

In a process for preparing a divinylarene product, a liquid extractive agent, component (i), may be used in the process of the present invention for separating DVB from EVB, DEB and other non-desired components in a composition mixture; i.e., to preferentially enrich the DVB in the bottoms of a column to a greater extent than its relative volatility in the absence of an extractive agent would allow. The liquid extractive agent useful in the present invention may include for example, an extractive agent having a normal boiling point of greater than 200° C. in one embodiment, greater than 225° C. in another embodiment and greater than 250° C. in still another embodiment up to a boiling point of 350° C. Generally, the normal boiling point of the liquid extractive agent useful in the present invention may be from about 200° C. to about 350° C. in one embodiment, from about 225° C. to about 325° C. in another embodiment, and from about 250° C. to about 300° C. in still another embodiment.

In general, extractive agents that may be useful in the present invention may include for example ketones, amides with no hydrogen (H) atom on the nitrogen, secondary amines, ethers, oxides, sulfoxides, and mixtures thereof. Ionic liquids such as 1-ethyl-3-methylimidazolium trifluoroacetate may also be used as extractive agents although these extractive agents are currently expensive which detracts from their commercial feasibility.

As one mechanism for selecting the extractive agent to be used in the present invention process, vapor-liquid equilibrium measurements may be useful in determining the effect of the various extractive agents on, for example, the EVB-DVB relative volatility. In one preferred embodiment, the liquid extractive agent useful in the present invention can include for example, sulfolane (2,3,4,5-tetrahydrothiophene-1,1-dioxide), triethylene glycol (TEG), N-formylmorpholine (NFM), tripropylene glycol, acetamide, or any combination thereof, as indicated by liquid-liquid equilibrium results. For example, based on vapor-liquid equilibria calculated from liquid-liquid equilibrium results for sulfolane/n-octane/EVB/DVB, the relative volatility between EVB and DVB can be boosted to about 2.4 to about 2.8 with sulfolane as the extractive agent when operating, for example, in the range of from about 95° C. to about 115° C. range at a tower top pressure of 12 mmHg. The use of an extractive agent that results in a larger boost of the EVB-DVB relative volatility than an alternative different extractive agent can be advantageous in the selection process of selecting an extractive agent for the separation of DVB from EVB in accordance with the present invention.

Other factors which may be considered as part of the selection criteria for an extractive agent useful in the present invention can include, but is not limited to, for example, the extractive agent's: low procurement cost, availability in large quantities, thermal stability, low viscosity at process temperatures, low toxicity, and low freezing point for ease of handling. One or more of the above qualities may be found in one compound or a mixture of compounds used as the extractive agent. Depending on the various qualities desired for the extractive agent used, the selection of an extractive agent may be governed by a choice of which combination of the above factors are more favored than another combination of the above factors.

In another embodiment, some of the extractive agents useful in the present invention may be extractive agents which have been modified with small quantities of various additives. For instance, an extractive agent such as sulfolane may be mixed with a small percentage of water, typically in the range of about 2-3 wt %. This small amount of water can be added to the sulfolane to reduce the freezing point of the sulfolane from an initial freezing point of about 27-28° C. to a freezing point of about 10° C. The above small amount of added water does not deleteriously affect the characteristics/properties of sulfolane as an extractive agent for DVB-EVB separation.

Generally, the ratio of liquid extractive agent to DVB/EVB/DEB feed used in the process may be for example, from 0.5 to about 10 in one embodiment, from about 1.0 to about 7.0 in another embodiment; from about 1.25 to about 5.0 in still another embodiment; and from about 1.5 to about 3.0 in yet another embodiment. Below a ratio of about 0.5, the concentration of the liquid extractive agent in the distillation tower is insufficient to affect the EVB/DVB relative volatility significantly, and above a ratio of about 10, an excessive amount of liquid extractive agent has to be passed through a distillation tower and requires a large extractive distillation tower and downstream processing equipment to recover the extractive agent for reuse, rendering the overall process uneconomical.

Another step of the process of the present invention includes recovering the DVB once the DVB is extracted from the EVB. Generally, any conventional recovery process can be used in the DVB recovering step such as for example (i) removing (e.g. by distillation) the DVB from the extractive agent and other “heavy” compounds.

Other optional steps that can be included as part of the present invention process can include for example (ii) optionally, removing the extractive agent from the DVB by a liquid-liquid extraction process, e.g., by water washing; (iii) removing the extractive agent from the other “heavy” compounds (e.g., tars, inhibitors, polymers, oligomers and other undesirable by-products); (iv) recycling the extractive agent to the extractive distillation process or to a further processing operation of the extractive agent; and (v) providing a rectifying means to hold down (remove) the extractive agent from the vapors at the top of the extractive distillation tower (e.g., adding extra height (packing) in a single column or in a separate additional distillation column).

The DVB product is a mixture of meta- and para-DVB and meta- and para-EVB (and small amounts of ortho-EVB). This DVB product can advantageously be obtained by the process of the present invention in various grades. For example, by varying the extractive agent-to-feed ratio, the reflux ratio, and the overall bottoms-to-feed ratio in the extractive distillation tower, the DVB-to-EVB ratio of the DVB product can be manipulated to achieve for example, DVB:EVB ratios of 40:60, 55:45, 63:37, 80:20, 95:5, or even higher. The residual DEB concentration in the DVB product may also be adjusted by varying the same process parameters as well as designing the extractive distillation tower with greater or fewer theoretical equilibrium stages.

In one embodiment, the concentration of the divinylarene in the divinylarene product stream of the present invention can be generally from about 40 wt % to about 99.9 wt % divinylarene; from about 50 wt % to about 99.9 wt % divinylarene in another embodiment; from about 55 wt % to about 99.9 wt % divinylarene in still another embodiment; from about 60 wt % to about 99.9 wt % divinylarene in yet another embodiment; and from about 80 wt % to about 99.9 wt % divinylarene in even yet another embodiment. The concentration of the divinylarene in the divinylarene product stream can be as low as about 40 wt %; and in one preferred embodiment, for example, the concentration of the divinylarene in the divinylarene product stream can be at least about 50 wt %.

The process of present invention, and/or any of the steps thereof, may be carried out in a batch, a semi-batch or a continuous process. In order to carry out the process, a novel apparatus is used in the above process as described herein. Thus, another broad embodiment of the present invention is directed to an apparatus for continuously separating DVB from EVB.

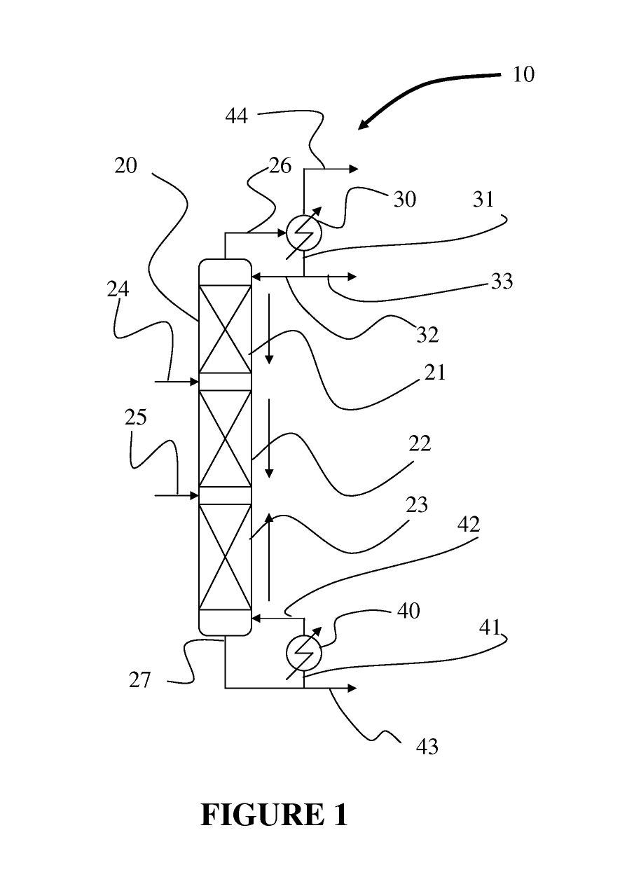

In one general embodiment for example, the apparatus of the present invention may include at least one distillation tower; and ancillary equipment as shown in FIG. 1.

With reference to FIG. 1, there is shown a DVB extractive distillation apparatus and flow diagram showing a distillation apparatus, generally indicated by numeral 10, with incoming feed stream and outgoing streams. The distillation apparatus 10 includes a distillation tower 20 comprising a combination of at least three bed sections such as for example a top bed 21, a middle bed 22, and a bottom bed 23, with a condensing exchanger apparatus 30 and a reboiler apparatus 40. An extractive agent feed stream 24 enters the tower 10 near the top of the tower and a crude DVB composition feed stream 25 enters the tower 10 near the bottom of the tower 10 and near the bottom of the middle packed bed section 22. A vapor stream 26 exits the tower 10 at the top of the tower 10 and a liquid heavies stream 27 exits the tower 10 at the bottom of the tower 10. Any one of the streams 26, 27, 31, 33, 43, and/or 44 exiting the tower 10 can be stored or processed further in subsequent operating units (not shown).

In the DVB extractive distillation, the crude DVB feed stream 25 which has had light impurities (for example, benzene, toluene, styrene, ethyl toluene, vinyl toluene) removed is fed to the tower 20 near the top of the bottom packed bed section 23 in the tower. The extractive agent stream 24 is fed to the tower 20 near the top of the middle packed bed section 22 in the tower. Vapor exits the top of the tower in stream 26 and stripped heavies exit the bottom of the tower in stream 27.

The vapor stream 26 exiting the top of the tower passes through the condenser 30 and the condensed stream 31 exiting the condenser can be split up into two streams—a reflux stream 32 and a liquid distillate stream 33 which can be sent to another operating unit for further processing. The small vapor distillate stream 44 exiting the condenser 30 consists of non-condensable gases and small quantities of uncondensed components, mainly lights.

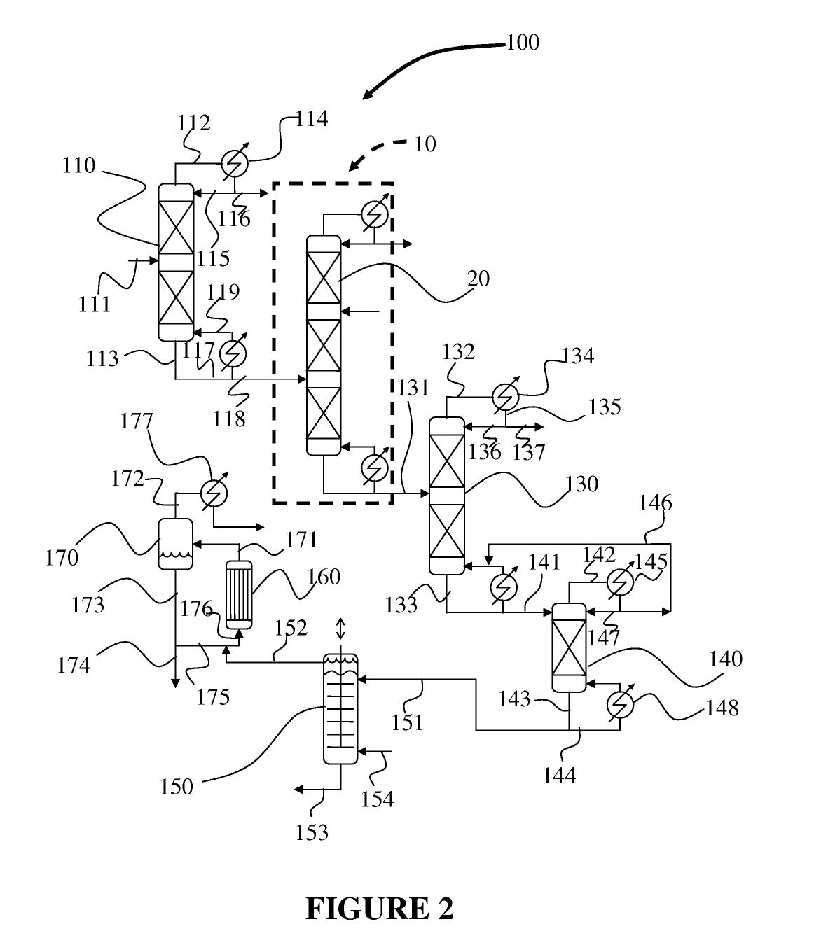

The bottom heavies stream 27 exiting the bottom of the tower can be split up into two streams—one stream 41 can be passed through the reboiler 40 and the heated stream 42 exiting the reboiler can be recycled to the tower 20. Another liquid stream 43 split from stream 27 is a product stream 43 which can be sent to another operating unit for further processing such as for example a finishing operation as shown in the process flow diagram of FIG. 2.

During the extractive distillation, the extractive agent preferentially absorbs DVB out of the vapor flowing in an upwardly direction through the middle bed 22 and carries the absorbed DVB in a downwardly direction in the tower in a liquid to exit at the bottom of the tower. Likewise, EVB is preferentially stripped out of the liquid in the bottom bed 23 to the desired DVB/EVB ratio of the final product. The top packed bed 21 is used to hold down the extractive agent out of the distillate containing predominantly DEB and EVB. In the tower 10, the ratio of the bottoms draw-off rate to the crude DVB feed rate can be adjusted to control the DVB/EVB ratio in the bottoms stream 27, the ratio of the extractive agent feed rate to the crude DVB feed rate can be adjusted to control the DVB recovery out of the crude DVB feed, and the reflux ratio can be adjusted to control the concentration of the extractive agent carried overhead with the distillate.

The bottoms product stream 43 from the extractive distillation tower is fed to another distillation tower to separate purified DVB as distillate from the extractive agent and heavy impurities such as naphthalene, inhibitors, oligomers, and polymers. This final distillation to produce an overhead DVB product is similar to a finishing distillation since the extractive agents being considered have vapor pressures in the same range as that of the flux oil which may be added to a distillation train to keep the heavy bottoms stream from becoming too viscous and causing plugging problems. However, in the process of the present invention, the addition of a separate flux oil to the extractive distillation process is not necessary. If needed to keep the temperatures in the bottom of the tower low enough, this final distillation tower may be split up into two towers, each with its own condenser and reboiler to allow a fresh vacuum to be pulled on the second “bottom” tower of the series.

In general, the extractive agent in the bottoms stream from a finishing tower is preferably recovered and recycled back to an extractive distillation tower for economical process operation. One way to accomplish this is with a water wash of the bottoms to extract the more-polar extractive agent from the less-polar naphthalene, oligomers, and polymer Inhibitors may partition between the two phases because of their mildly polar makeup. Although a single stage wash may be sufficient, a multi-stage wash may be more preferred to keep the water usage low. An evaporation of water from the recovered extractive agent with recycle of the evaporated water back to the water wash step advantageously may dry the extractive agent for reuse in the extractive distillation.

In one embodiment, when sulfolane is used as the extractive agent, and since sulfolane normally and preferably has a small amount of water content to prevent freezing issues with pure sulfolane, a sharp separation of sulfolane from water may not be necessary for the reuse of sulfolane. In another embodiment, if evaporation does not result in a particular extractive agent having a sufficiently low water content to be recycled to the extractive distillation operation, a distillation may be used in place of the water evaporator.

With reference to FIG. 2, there is shown a process flow diagram of an extractive distillation process with equipment of the present invention, generally indicated by numeral 100. The process 100 incorporates a DVB extractive distillation apparatus, generally indicated with a dotted line as numeral 10 (similar to the apparatus 10 shown in FIG. 1). In FIG. 2, there is shown a process flow diagram including for example a distillation lights tower 110, an extractive distillation tower 20, a finishing tower 130, a tar tower 140, a water wash tower 150, a water evaporation means 160, and a condensing/separation means 170.

The process shown in FIG. 2 includes the extractive distillation tower 20 described above with reference to FIG. 1 and enclosed generally in dotted lines in FIG. 2. The feed stream to the tower 20 is essentially the exit stream 113,118 from the distillation lights tower 110. A crude DVB stream is fed into tower 110 via feed stream 111 and a vapor stream 112 exits the tower 110 at near the top of the tower 110; and a bottom stream 113 exits the tower 110 at near the bottom of the tower 110.

The feed stream 131 to the finishing tower 130 is essentially the bottoms stream 27,43 of the extractive distillation tower 20. A vapor stream 132 from the finishing tower 130 exits the tower 130 at near the top of the tower 130 and fed into a condenser 134; and at least a portion of the stream 132 may be condensed to generally form a DVB product stream 137 exiting the tower 130. A bottoms stream 133 exiting the tower 130 contains a certain quantity of extractive agent, DVB, impurities, and heavies. The vapor stream 132 from the finishing tower 130 is preferably condensed via a condenser means 134 and a portion of the condensed DVB product stream 135 can be recycled to the finishing tower via stream 136 and a portion of the condensed DVB product stream 135 exits the finishing tower via product stream 137 at a desirable purity level, for instance about 95 wt %.

The feed stream 141 to the tar tower 140 is essentially the bottoms stream 133, 141 of the finishing tower 130. A vapor stream 142 from the tar tower 140 contains solvent or impurities such as naphthalene and a certain quantity of DVB which can be recycled back to the finishing tower 130; and a bottoms stream 143 exiting the tar tower 140 contains a certain quantity of extractive agent and tars.

A portion of the bottom stream 143 may be preferably sent as a feed stream 151 to the water wash tower 150 to separate wet extractive agent via stream 152 at near the top of the tower 150 from a tar stream via stream 153 near the bottom of the tower 150. The tar stream 153 can be sent to a treatment process or another further operation (not shown). The wet extractive agent stream 152 can be sent to a water evaporation means 160. In one embodiment, the evaporator apparatus 160 functions to form a vapor stream 171 which may be fed into a condensing/separation means 170.

In one embodiment, the stream 152 may be mixed with a recycle stream 173, 175 from the condensing means 170 to form feed stream 176 to the evaporator 160. Use of the apparatuses 160 and 170 can be a preferred means to further process the wet extractive agent 152 leaving the tower 150 for example to separate the extractive agent from water. The extractive agent separated from water exits the condensing means 170 as stream 173; and the water separated from the extractive agent exits the condensing means 170 via a vapor stream 172. The vapor stream 172 may be condensed in a means 177 and the water in stream 172 can be recycled to another unit operation, stored, or otherwise further processed (not shown).

A portion of the extractive agent stream 173 can be recycled via stream 175 back to the evaporator 160 and eventually returned to the condensing means 170 via stream 171. A portion of the extractive agent stream 173 via stream 174 can be recycled to another unit operation, stored, or otherwise further processed (not shown).

### EXAMPLES

The following examples further illustrate the present invention in detail but are not to be construed to limit the scope thereof.

Various terms and designations used in the following examples are explained herein below:

“DVB” stands for divinylbenzene and includes a mixture of the meta and para isomers of divinylbenzene.

“EVB” stands for ethylvinylbenzene and includes a mixture of the ortho, meta, and para isomers of ethylvinylbenzene.

“DEB” stands for diethylbenzene and includes a mixture of the ortho, meta, and para isomers of diethylbenzene.

“Relative volatility” is an indication of the ease or difficulty of separating two components by distillation and is defined as the ratio of the concentrations of two components in the vapor phase divided by the ratio of the same components in the liquid phase at thermal and mass transfer equilibrium.

The standard analytical equipment and methods used in the Example includes: gravimetric analysis and gas chromatography with flame ionization detector.

### Example 1

In this Example 1, several laboratory experiments were carried out including providing a mixture of extractive agent, DEB, EVB, and DVB; and boiling the mixture under vacuum in a single-stage vapor-liquid equilibrium (VLE) still to measure vapor and liquid compositions at equilibrium and determine the resulting relative volatilities of the components in the mixture. These relative volatilities were compared to relative volatilities resulting from similar experiments using a mixture of DEB, EVB, and DVB in the absence of an extractive agent to determine the degree of enhancement resulting from the use of the extractive agent. The results of these experiments are described in the following Tables I-VI:

TABLE I
DEB/EVB/DVB VLE Results - No Extractive Agent

| P | T | | Composition (wt %) | | | | | | |
|---|---|---|---|---|---|---|---|---|---|
| (mmHg) | (° C.) | Phase | DEB | mEVB | pEVB | mDVB | pDVB | Naphthalene | Total |
| 10 | 68.719 | Liquid | 32.87 | 26.76 | 7.76 | 21.72 | 10.46 | 0.44 | 100.00 |
| | | Vapor | 46.50 | 25.59 | 6.94 | 14.62 | 6.17 | 0.18 | 100.00 |
| 20 | 82.289 | Liquid | 32.11 | 26.76 | 7.83 | 22.13 | 10.71 | 0.47 | 100.00 |
| | | Vapor | 45.37 | 25.80 | 7.06 | 15.15 | 6.43 | 0.19 | 100.00 |

TABLE II
DEB/EVB/DVB Relative Volatility
Calculation - No Extractive Agent

| | | | Composition (wt %) | | | | |
|---|---|---|---|---|---|---|---|
| P | T | | | | | Naph- | |
| (mmHg) | (° C.) | Phase | DEB | EVB | DVB | thalene | Total |
| 10 | 68.719 | Liquid | 32.87 | 34.52 | 32.18 | 0.44 | 100.00 |
| | | Vapor | 46.50 | 32.53 | 20.79 | 0.18 | 100.00 |
| | | RV | 2.19 | 1.46 | 1.00 | | |
| 20 | 82.289 | Liquid | 32.11 | 34.59 | 32.84 | 0.47 | 100.00 |
| | | Vapor | 45.37 | 32.85 | 21.58 | 0.19 | 100.00 |
| | | RV | 2.15 | 1.45 | 1.00 | | |

TABLE III
Sulfolane 1.5 to 1 DEB/EVB/DVB VLE Results

| P | T | | Composition (wt %) | | | | | | | |
|---|---|---|---|---|---|---|---|---|---|---|
| (mmHg) | (° C.) | Phase | DEB | mEVB | pEVB | mDVB | pDVB | Naphthalene | Sulfolane | Total |
| 10 | 73.45 | Liquid | 10.32 | 9.32 | 2.72 | 8.00 | 3.86 | 0.16 | 65.62 | 100.00 |
| | | Vapor | 52.76 | 23.66 | 6.67 | 10.50 | 4.51 | 0.10 | 1.80 | 100.00 |
| 20 | 86.21 | Liquid | 9.69 | 9.06 | 2.65 | 7.93 | 3.83 | 0.16 | 66.68 | 100.00 |
| | | Vapor | 51.37 | 23.96 | 6.74 | 11.04 | 4.75 | 0.11 | 2.04 | 100.00 |

TABLE IV
Sulfolane 1.5 to 1 DEB/EVB/DVB Relative Volatility Calculation

| P | T | | Composition (wt %) | | | | | |
|---|---|---|---|---|---|---|---|---|
| (mmHg) | (° C.) | Phase | DEB | EVB | DVB | Naphthalene | Sulfolane | Total |
| 10 | 73.45 | Liquid | 10.32 | 12.04 | 11.86 | 0.16 | 65.62 | 100.00 |
| | | Vapor | 52.76 | 30.33 | 15.01 | 0.10 | 1.80 | 100.00 |
| | | RV | 4.04 | 1.99 | 1.00 | | | |
| 20 | 86.21 | Liquid | 9.69 | 11.71 | 11.75 | 0.16 | 66.68 | 100.00 |
| | | Vapor | 51.37 | 30.70 | 15.79 | 0.11 | 2.04 | 100.00 |
| | | RV | 3.95 | 1.95 | 1.00 | | | |

TABLE V
Sulfolane 2.5 to 1 DEB/EVB/DVB VLE Results

| P | T | | Composition (wt %) | | | | | | | |
|---|---|---|---|---|---|---|---|---|---|---|
| (mmHg) | (° C.) | Phase | DEB | mEVB | pEVB | mDVB | pDVB | Naphthalene | Sulfolane | Total |
| 10 | 74.97 | Liquid | 6.30 | 6.16 | 1.73 | 5.52 | 2.66 | 0.12 | 77.51 | 100.00 |
| | | Vapor | 51.78 | 23.90 | 6.79 | 10.46 | 4.53 | 0.10 | 2.44 | 100.00 |
| 20 | 86.83 | Liquid | 5.79 | 5.92 | 1.68 | 5.43 | 2.63 | 0.12 | 78.43 | 100.00 |
| | | Vapor | 50.51 | 24.15 | 6.83 | 10.98 | 4.76 | 0.11 | 2.67 | 100.00 |

TABLE VI
Sulfolane 2.5 to 1 DEB/EVB/DVB Relative Volatility Calculation

| P | T | | Composition (wt %) | | | | | |
|---|---|---|---|---|---|---|---|---|
| (mmHg) | (° C.) | Phase | DEB | EVB | DVB | Naphthalene | Sulfolane | Total |
| 10 | 74.97 | Liquid | 6.30 | 7.89 | 8.18 | 0.12 | 77.51 | 100.00 |
| | | Vapor | 51.78 | 30.69 | 14.99 | 0.10 | 2.44 | 100.00 |
| | | RV | 4.49 | 2.12 | 1.00 | | | |
| 20 | 86.83 | Liquid | 5.79 | 7.60 | 8.06 | 0.12 | 78.43 | 100.00 |
| | | Vapor | 50.51 | 30.98 | 15.73 | 0.11 | 2.67 | 100.00 |
| | | RV | 4.47 | 2.09 | 1.00 | | | |

The above experiments show that the relative volatility of EVB to DVB in the absence of an extractive agent is only about 1.5; and that the relative volatility of EVB to DVB in the presence of an extractive agent such as sulfolane under similar conditions of temperature is enhanced to about 2.0-2.1. Thus, the above experiments indicate that DVB/EVB separation by distillation can be facilitated using an extractive agent such as sulfolane.

1. An extractive distillation process for extracting divinylarene from a composition mixture stream comprising at least an ethylvinylarene and at least a divinylarene, the process comprising the steps of:
(a) contacting a composition mixture stream comprising at least an ethylvinylarene and at least a divinylarene with a liquid extractive agent such that at least a portion of the divinylarene present in the composition mixture is extracted into the liquid extractive agent; and
(b) recovering at least a portion of the divinylarene extracted from the ethylvinylarene stream to form a divinylarene product stream; wherein the divinylarene product stream contains at least 50 weight percent divinylarene.

2. The process of claim 1, wherein the liquid extractive agent comprises triethylene glycol, sulfolane, N-formylmorpholine, tripropylene glycol, acetamide, or mixtures thereof.

3. The process of claim 1, wherein the divinylarene comprises divinylbenzene.

4. The process of claim 1, wherein the ethylvinylarene comprises ethylvinylbenzene.

5. The process of claim 1, wherein the contacting step is carried out at a temperature of from about 45° C. to about 160° C.; a pressure of from about 0.006 atmosphere to about 0.07 atmosphere; and with a number of theoretical equilibrium stages of from about 5 to about 100.

6. The process of claim 1, wherein the contacting step is carried out in a distillation tower.

7. An apparatus for extracting divinylarene from an ethylvinylarene comprising:
(A) a means for contacting a composition mixture stream comprising at least an ethylvinylarene and at least a divinylarene with a liquid extractive agent such that at least a portion of the divinylarene present in the composition mixture is extracted into the liquid extractive agent; and
(B) a means for recovering at least a portion of the divinylarene extracted from the ethylvinylarene stream to form a divinylarene product stream; wherein the divinylarene product stream contains at least 50 weight percent divinylarene.

8. An apparatus for extracting divinylarene from an ethylvinylarene comprising:
(A) a means for contacting an ethylvinylarene stream containing divinylarene with a liquid extractive agent such that at least a portion of the divinylarene present in the ethylvinylarene stream is extracted into the liquid extractive agent;
(B) a means for recovering the divinylarene extracted from the ethylvinylarene connected to the contacting means of (a); and
(C) a means for recovering the extractive agent from the ethylvinylarene of means (b) which has been depleted of divinylarene.
